(19) United States
(12) Patent Application Publication
(10) Pub. No.: US 2003/0163107 A1
(43) Pub. Date: Aug. 28, 2003

(54) COLORED PANTY LINERS

(76) Inventor: Mr. Bernard Daskal, New York, NY (US)

Correspondence Address: Charles Macedo, Esq., Brian Comack, Esq., Amster, Rothstein & Ebenstein, 90 Park Avenue, New York, NY, 10016, US, ptodocket@arelaw.com, 212-697-5995, 212-286-0854

(21) Appl. No.: 09/489,655

(22) Filed: Jan. 24, 2000

(51) Int. Cl. A61F 13/15

(52) U.S. Cl. 604/385.01

(57) ABSTRACT

### Abstract of the Disclosure

A feminine hygiene pad that is worn in the crotch portion of an undergarment. A central body fluid absorbent core has a body facing side and an undergarment facing side. A fluid permeable topsheet is overlaid on the body facing side of the central body fluid absorbent core and has a dark color, which may be black, brown, or red. A fluid impermeable backsheet is overlaid on the undergarment facing side of the central body fluid absorbent core and placed in register with the fluid permeable topsheet.

### Background of the Invention

### Field of the Invention

This invention relates generally to disposable absorbent articles and, in particular, to feminine hygiene pads, such as sanitary napkins, catamenial pads, panty liners, and the like, that are designed to absorb discharged body fluids.

A variety of feminine hygiene pads and thin, absorbent panty liners are available on the market today for use between menstrual cycles and, in conjunction with other feminine hygiene products, during menstruation. Panty liners and other feminine hygiene pads provide the user with protection from undergarment staining. These products are worn in the crotch portion of an undergarment. They are typically comprised of a fluid permeable topsheet, an absorbent body, and a fluid impermeable backsheet for preventing the absorbed fluid from striking through onto the surface of the undergarment. When used properly, the fluid permeable topsheet faces the skin and the fluid impermeable backing faces the undergarment.

It is known in the art to color various portions of feminine hygiene pads. For example, U.S. Patent No. 5,261,899 to Visscher et al. ("the Visscher '899 Patent") is directed to polymeric multilayer films having high concentrations of fillers to increase the opacity of the film. Such multilayer films are used in the construction of topsheets for sanitary napkins. In Fig. 3, the Visscher '899 Patent discloses a three-layer topsheet 120 for a sanitary napkin. The topsheet 120 includes outer layers 122 and a central filler-containing layer 124, which contains a high percentage of opacifying fillers 126. In Col. 5, at lines 37-40, the Visscher '899 Patent teaches that useful fillers 126 include colored pigments such as carbon black, etc. However, the colored fillers 126 disclosed in the Visscher '899 Patent are used in the central layer 124 of the topsheet 120, and not in either of the outer layers 122.

The Visscher '899 Patent also teaches that it is possible to construct the three-layer topsheet such that each of the three layers are unique. By way of example, the Visscher '899 Patent describes that the outer layers 122 of the topsheet may contain different colored pigments. Thus, it is possible to produce a multilayer film 120 with each side of the film having a different color, if a first colored pigmentation is employed in one of the outer layer 122, and white or a second colored pigmentation is employed in the other of the outer layers 122. Nonetheless, the Visscher '899 Patent does not indicate the desirability of any particular color for the topsheet.

U.S. Patent No. 5,961,505 to Coe, et al. ("the Coe '505 Patent") discloses, in Fig. 1, a sanitary napkin 10 which has a multi-layered construction. The napkin 10 has a bicomponent cover 24. Underneath the cover 24 is a separation means 18, which functions to direct body fluid downward into an absorbent 12. The separation means 18 is shown is Fig. 1 as consisting of two layers 20 and 22. In Col. 7, lines 20-26, the Coe '505 Patent teaches that the cover 24 should be white or opaque to indicate cleanliness. The separation means 18, however, can be colored, and the Coe '505 Patent suggests peach and blue as two colors which are well received by consumers, presumably because of the aesthetic appeal of those colors. The Coe '505 Patent states that coloring the separation means 18 assists in masking the stains, especially blood stains, which may form as the body fluid dries on the absorbent 12. Nonetheless, the Coe '505 Patent teaches coloring the separation means 18, and not the cover 24.

U.S. Patent No. 4,673,403 to Lassen et al. ("the Lassen '403 Patent") is directed to providing a sanitary napkin with a means for insuring proper placement of the napkin. As stated in the Description Of The Background Art section of the Lassen '403 Patent, in Col. 1, at lines 42-48, the purpose of the invention disclosed therein is to indicate to the user of the napkin when leakage was caused by improper placement of the napkin on the user's body, as opposed to being caused by a failure of the napkin. In Fig. 11, the target area 150 of the napkin 30 is placed below the vaginal orifice for best pad placement. In Col. 14, at lines 22-52, the Lassen '403 Patent teaches the use of a colorant layer, for example on the cover (i.e. topsheet) of the napkin, to identify the target area 150. In Col. 14, at lines 32-38, the Lassen '403 Patent further teaches the particular desirability of the color blue. By studying the staining of the target area 150 after use of the napkin 30, the user is able to determine whether the napkin 30 was properly placed for her body. Thus, one who examines the target area 150 of a sanitary napkin according to the invention of the Lassen '403 Patent is able to discern the presence of body fluid on the colored target area 150 of the napkin.

The art clearly teaches the desirability of using whitish pigmentation in the topsheets of feminine hygiene pads. For example, in Col. 7, lines 20-26, the Coe '505 Patent teaches that the cover of the sanitary napkin disclosed therein should be white or opaque to indicate cleanliness. U.S. Patent No. 5,006,394 to Baird ("the Baird '394 Patent") discloses a multilayer polymeric film for use in the topsheets of catamenial pads. In Col. 3, lines 19-27, the Baird '394 Patent teaches the desirability of a whitish appearance for the film if the film is to be used in disposable consumer products, such as catamenial pads. Furthermore, the fluid permeable topsheets in commercially available panty liners, such as those sold under the FREEDOM LIGHTDAYS, KOTEX LIGHTDAYS, ALLWAYS ALLDAYS, CAREFREE, DUANE READE and RITE AID brand names, are white or off-white.

Taharat Hamishpachah is the Hebrew term for a set of millennial-old Jewish laws and customs regarding sexual relations. Among its practitioners, who are mostly orthodox Jews, its observance is sacred. At the heart of the practice is a simple rule against coitus whenever a woman experiences bleeding from her uterus, whether during her normal menstrual period or at any other time. The limitation on coitus lasts for a statutory "menstrual period" (lasting the entire period of menstruation, but in any event not less than 5 days) and the seven (7) days immediately after the cessation of menstruation (the "reserved period"). Taharat Hamishpachah also promotes fulfilling sexual intercourse outside the menstrual and reserved periods.

Practitioners of Taharat Hamishpachah scrutinize each vaginal discharge for indicia of uterine bleeding. If a question arises as to the nature of a particular discharge, a rabbi with an expertise in Taharat Hamishpachah is consulted. In such circumstances, coitus is prohibited until the expert rabbi can rule on the matter. If available, the expert rabbi will analyze a sample of the questionable discharge as it appears on a panty, tissue, or feminine-hygiene napkin, pad, panty liner, or other "garment".

There are numerous factors a rabbi considers in determining whether a discharge appearing on a garment outside the menstrual and reserved periods is attributable to uterine bleeding, including the size and color of the discharge. A dark stain of suitable size found on a white or off-white garment, such as white panties, any commercially-available panty-liner, or even white bedding, is presumptively attributable to uterine bleeding. However, the same stain on a colored garment is presumptively not attributable to uterine bleeding.

For this reason, female practitioners of Taharat Hamishpachah facilitate the uninterrupted feasibility of sexual relations by wearing colored panties and sleeping on colored or darkly patterned sheets outside the menstrual and reserved periods. Because the topsheet of commercially-available panty liners and feminine hygiene pads is white or off-white, they are generally regarded by female practitioners of Taharat Hamishpachah as not acceptable outside the menstrual and reserved periods. This is not to say that the laws of Taharat Hamishpachah forbid their use, only that practitioners of Taharat Hamishpachah have two difficult choices outside the menstrual and reserved periods: wear commercially-available panty liners or feminine hygiene pads and risk a determination that a dark discharge found on such garments is attributable to uterine bleeding, or do not wear them and risk staining their clothing. Consequently, there is a need for a commercially available feminine hygiene pad that has a darkly colored topsheet.

### Brief Summary of the Invention

The present invention satisfies the need for feminine hygiene pads, such as sanitary napkins, catamenial pads, panty liners, and the like, that have darkly colored topsheets.

In accordance with an embodiment of the present invention, a feminine hygiene pad that is worn in the crotch portion of an undergarment comprises: a central body fluid absorbent core having a body facing side and an undergarment facing side; a fluid permeable topsheet overlaid on the body facing side of the central body fluid absorbent core and having a dark color; and a fluid impermeable backsheet overlaid on the undergarment facing side of the central body fluid absorbent core and placed in register with the fluid permeable topsheet. The dark color may comprise black, brown, or red.

The above, and other features, aspects, and advantages of the present invention will become apparent from the following description.

### Description of the Preferred Embodiments

A feminine hygiene pad comprises a fluid permeable topsheet, a central body fluid absorbent core, and a fluid impermeable backsheet for preventing the fluid that is absorbed by the absorbent body from striking through onto the surface of the undergarment in which the feminine hygiene pad is worn. The central body fluid absorbent core has a body facing side and an undergarment facing side. The topsheet is overlaid on the body facing side of the central body fluid absorbent core, and the backsheet is overlaid on the undergarment facing side of the central body fluid absorbent core. The topsheet and backsheet are placed in register on opposed faces of the absorbent core, and then are wrapped using known techniques to enclose the core so that the topsheet and backsheet are affixed to, or otherwise associated with, opposed faces of the absorbent core. The seams formed by the junctures of the topsheet and backsheet are sealed using any known means, including but not limited to adhesive bonding, ultrasonic welding, or crimping, that are suitable for providing a fluid-tight seam between the topsheet and the backsheet. The size of the absorbent core is determined by the expected amount of vaginal discharge. For example, for a panty liner, the size of the absorbent core is reduced, as desired, to accommodate lesser amounts of vaginal discharge. The sizes of the topsheet and backsheet are adjusted accordingly to accommodate the size of the absorbent core.

According to a preferred embodiment of the present invention, the topsheet of the feminine hygiene pad is darkly colored. In preferred embodiments of the present invention, the color of the topsheet can be black, brown, or red. The desired dark coloration of the topsheet is achieved by providing the topsheet with an appropriate concentration of colored pigments using known techniques.

After the feminine hygiene pad is assembled in the manner described above, the pad is placed in the crotch portion of the undergarment of the wearer with the fluid permeable topsheet facing the skin and the fluid impermeable backsheet oriented toward the crotch portion of the undergarment. Vaginal discharge or menses will be deposited on the fluid permeable topsheet and will drain to the absorbent core. The discharge or menses will be retained in the core by the fluid impermeable backsheet.

Having described preferred embodiments of the invention, it is to be understood that the invention is not limited to those precise embodiments, and that various changes and modifications may be effected therein by one skilled in the art without departing from the scope or spirit of the invention as defined in the appended claims.

What is Claimed is:

1. A feminine hygiene pad that is worn in the crotch portion of an undergarment, said feminine hygiene pad comprising:
a central body fluid absorbent core having a body facing side and an undergarment facing side;
a fluid permeable topsheet overlaid on said body facing side of said central body fluid absorbent core and having a dark color; and
a fluid impermeable backsheet overlaid on said undergarment facing side of said central body fluid absorbent core and placed in register with said fluid permeable topsheet.

2. A feminine hygiene pad that is worn in the crotch portion of an undergarment, said feminine hygiene pad comprising:
a central body fluid absorbent core having a body facing side and an undergarment facing side;
a fluid permeable, black topsheet overlaid on said body facing side of said central body fluid absorbent core; and
a fluid impermeable backsheet overlaid on said undergarment facing side of said central body fluid absorbent core and placed in register with said fluid permeable topsheet.

3. A feminine hygiene pad that is worn in the crotch portion of an undergarment, said feminine hygiene pad comprising:
a fluid permeable, brown topsheet overlaid on said body facing side of said central body fluid absorbent core; and
a fluid impermeable backsheet overlaid on said undergarment facing side of said central body fluid absorbent core and placed in register with said fluid permeable topsheet.

4. A feminine hygiene pad that is worn in the crotch portion of an undergarment, said feminine hygiene pad comprising:
a central body fluid absorbent core having a body facing side and an undergarment facing side;
a fluid permeable, red topsheet overlaid on said body facing side of said central body fluid absorbent core; and
a fluid impermeable backsheet overlaid on said undergarment facing side of said central body fluid absorbent core and placed in register with said fluid permeable topsheet.
